SEQUENCE LISTING (35 sequences)

SEQ ID NO: 1 - DNA, 1946 bp, Artificial sequence
  The sequence is synthesized.
ttcattttctctcttggccactatttttttttttaattcccctttatctctcgattcgacatgggtgaactcaaggaaatcttgaaacagaggtatcatgagttgcttgactggaatgtcaaagcccctcatgtccctctcagtcaacgactgaagcattttacatggtcttggtttgcatgtactatggcaactggtggtgttggtttgattattggttctttcccctttcgattttatggtcttaatacaattggcaaaattgtttatattcttcaaatctttttgttttctctctttggatcatgcatgctttttcgctttattaaatatccttcaactatcaaggattcctggaaccatcatttggaaaagcttttcattgctacttgtcttctttcaatatccacgttcatcgacatgcttgccatatacgcctatcctgataccggcgagtggatggtgtgggtcattcgaatcctttattacatttacgttgcagtatcctttatatactgcgtaatggctttttttacaattttcaacaaccatgtatataccattgaaaccgcatctcctgcttggattcttcctattttccctcctatgatttgtggtgtcattgctggcgccgtcaattctacacaacccgctcatcaattaaaaaatatggttatctttggtatcctctttcaaggacttggtttttgggtttatcttttactgtttgccgtcaatgtcttacggttttttactgtaggcctggcaaaaccccaagatcgacctggtatgtttatgtttgtcggtccaccagctttctcaggtttggccttaattaatattgcgcgtggtgctatgggcagtcgcccttatatttttgttggcgccaactcatccgagtatcttggttttgtttctacctttatggctatttttatttggggtcttgctgcttggtgttactgtctcgccatggttagctttttagcgggctttttcactcgagcccctctcaagtttgcttgtggatggtttgcattcattttccccaacgtgggttttgttaattgtaccattgagataggtaaaatgatagattccaaagctttccaaatgtttggacatatcattggggtcattctttgtattcagtggatcctcctaatgtatttaatggtccgtgcgtttctcgtcaatgatctttgctatcctggcaaagacgaagatgcccatcctccaccaaaaccaaatacaggtgtccttaaccctaccttcccacctgaaaaagcacctgcatctttggaaaaagtcgatacacatgtcacatctactggtggtgaatcggatcctcctagtagtgaacatgaaagcgtttaagcttgtatgcttttccttaatttttctataaatctgtgtgccctgctcttaataccattatagattaatcattttgaatcattctgtatctttattgtactactggtactaattttgcttagacatttttgctccttcttcttctttttgtttaaattatacataccaaaattttggactttgaataatggtaatttttggttgtcgtagtgttaaatatgtatgcgtcttgcatatgaatcacgacgaaggaatcaattaaaaaatcaatcctgtacataataaaattaagtttatttatttcattttatcggatttaatcgtctaaaatttatatcttggtcatccaagcttatatctctttctactcttatcagcagcacactttagttatggttatttgaaaacttgtgtataaattcctggttatagagaaaatgagtataagacaacaaaaaaaagcctagtcggcatgcgacatgtctcaaacatatctttggcgtattgatgagcatcttacacactcactatacgtaacaataaaattaagagggatttcatgacaaaag

SEQ ID NO: 2 - DNA, 1623 bp, Artificial sequence
  The sequence is synthesized.
atgcgtgcacatgaaattttaaacaatccttttttaaataaaggaacagcttttactatgaaagaacgtcaagaattggggttgattggtcttcttccaccaactgttcaaacaattgaggaacaagctgaacaaacttacgaacaatatttgacaaaaccatctgatttagaaaaacgtcatttcttgatggaaatttttaatacaaaccgtactttgttttactacttattcaacaaacatattgtagaatttaatccagttgtttatgatccaacaattgctgatacaattgaaaactacagtcatttgttcgtagatccacaatatgctgcttatcttgatattaaccaccctgaaaacattactgaaacattgaaaaatgcagcaggtgacagagaaattcgtcttattgttgtaactgatgctgaaggaatccttggtattggagactggggaactcaaggtgttgatatctcagttggtaaattaatgatttatacagccgcagcaggtattgatccagcgtctgtacttccagttgttattgatgcaggaacaaatagaaaagaacttttagaagatcatttgtatcttggaaatcatcaagaacgtatttacggtgatcaatactacagtttcgtcgatcaatttgtagaaactgcagaatcaattttccctaaattgtaccttcactgggaagatttcggacgttcaaatgctgcaacaattttaaataactacaaaacaaaaatcccaacatttaatgatgacattcaaggaactggtattgttgttttaggtggtattttcggatcacttgacattacaggtgaaaaattaactgatcaagtatatctttgctatggtggtggttcagccggtgcagggattgctggtcgtgttcatgctgaaatggttagtgaaggtctttctgaagaagaagcttacaaacatttcttcatgattgatcaacaaggtttactttttgatgatatggaagaccttacaccagctcaaaaaccatttgctaaaaaacgtgctgattataaagatgctggagatatgactgaccttcttaacgttgttaagacagtaaaaccaactattttagtaggaacttcaactaatccaggtgcctttacaaaagaagttgttgaagcaatgtgtgctaatacagaacgcccagtaatcttccctatctcaaatccaactaaaaaaatggaaactacagctgaacaagttattgagtggtctgatggaaaagcttttgtcgctactggtgttccttcaggaacaatcagctacaaaggtgttgattatcaaattggtcaagcaaataactcacttatctacccaggtttgggcttaggaatgttggcatctgaagcaaaacttttgacagatgaaatgatcggtgcagctgcacattcattgagcggtttagtagatccaggtaaaccaggtgctcctgttcttcctccatttgaatttgttgctgatgtatcaattaaagttgcagaagcagttgctaagaaagctcaagaacaaggtcttactgaatctaaagaaactgatatggctaaagcagttcgtgatcttaaatggtatccagagtactaa

SEQ ID NO: 3 - DNA, 1308 bp, Artificial sequence
  The sequence is synthesized.
atggactacaacaagagatcttcggtctcaaccgtgcctaatgcagctcccataagagtcggattcgtcggtctcaacgcagccaaaggatgggcaatcaagacacattaccccgccatactgcaactatcgtcacaatttcaaatcactgccttatacagtccaaaaattgagacttctattgccaccattcagcgtctaaaattgagtaatgccactgcttttcccactttagagtcatttgcatcatcttccactatagatatgatagtgatagctatccaagtggccagccattatgaagttgttatgcctctcttggaattctccaaaaataatccgaacctcaagtatcttttcgtagaatgggcccttgcatgttcactagatcaagccgaatccatttataaggctgctgctgaacgtggggttcaaaccatcatctctttacaaggtcgtaaatcaccatatattttgagagcaaaagaattaatatctcaaggctatatcggcgacattaattcgatcgagattgctggaaatggcggttggtacggctacgaaaggcctgttaaatcaccaaaatacatctatgaaatcgggaacggtgtagatctggtaaccacaacatttggtcacacaatcgatattttacaatacatgacaagttcgtacttttccaggataaatgcaatggttttcaataatattccagagcaagagctgatagatgagcgtggtaaccgattgggccagcgagtcccaaagacagtaccggatcatcttttattccaaggcacattgttaaatggcaatgttccagtgtcatgcagtttcaaaggtggcaaacctaccaaaaaatttaccaaaaatttggtcattgacattcacggtaccaagggagatttgaaacttgaaggcgatgccggcttcgcagaaatttcaaatctggtcctttactacagtggaactagagcaaacgacttcccgctagccaatggacaacaagctcctttagacccggggtatgatgcaggtaaagaaatcatggaagtatatcatttacgaaattataatgccattgtgggtaatattcatcgactgtatcaatctatctctgacttccacttcaatacaaagaaaattcctgaattaccctcacaatttgtaatgcaaggtttcgatttcgaaggctttcccaccttgatggatgctctgatattacacaggttaatcgagagcgtttataaaagtaacatgatgggctccacattaaacgttagcaatatctcgcattatagtttataa

SEQ ID NO: 4 - DNA, 1737 bp, Artificial sequence
  The sequence is synthesized.
tctaactgatctatccaaaactgaaaattacattcttgattaggtttatcacaggcaaatgtaatttgtggtattttgccgttcaaaatctgtagaattttctcattggtcacattacaacctgaaaatactttatctacaatcataccattcttataacatgtccccttaatactaggatcaggcatgaacgcatcacagacaaaatcttcttgacaaacgtcacaattgatccctccccatccgttatcacaatgacaggtgtcattttgtgctcttatgggacgatccttattaccgctttcatccggtgatagaccgccacagaggggcagagagcaatcatcacctgcaaacccttctatacactcacatctaccagtgtacgaattgcattcagaaaactgtttgcattcaaaaataggtagcatacaattaaaacatggcgggcacgtatcattgcccttatcttgtgcagttagacgcgaatttttcgaagaagtaccttcaaagaatggggtctcatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaccgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgtta

SEQ ID NO: 5 - DNA, 32 bp, Artificial sequence
  The sequence is synthesized.
cgcggatcctctaactgatctatccaaaactg

SEQ ID NO: 6 - DNA, 32 bp, Artificial sequence
  The sequence is synthesized.
acgcgtcgactaacgaacgcagaattttcgag

SEQ ID NO: 7 - DNA, 41 bp, Artificial sequence
  The sequence is synthesized.
gaattccagatctcctcgagttcattttctctcttggccac

SEQ ID NO: 8 - DNA, 43 bp, Artificial sequence
  The sequence is synthesized.
tctatcgcagatccctcgagcttttgtcatgaaatccctctta

SEQ ID NO: 9 - DNA, 39 bp, Artificial sequence
  The sequence is synthesized.
gaattccagatctcctcgagatgcgtgcacatgaaattt

SEQ ID NO: 10 - DNA, 45 bp, Artificial sequence
  The sequence is synthesized.
tctatcgcagatccctcgagttagtactctggataccatttaaga

SEQ ID NO: 11 - DNA, 29 bp, Artificial sequence
  The sequence is synthesized.
cggcccgggtctaactgatctatccaaaa

SEQ ID NO: 12 - DNA, 29 bp, Artificial sequence
  The sequence is synthesized.
cggcccgggtaacgaacgcagaattttcg

SEQ ID NO: 13 - DNA, 40 bp, Artificial sequence
  The sequence is synthesized.
ccgctaacaatacctgggccccagctgaagcttcgtacgc

SEQ ID NO: 14 - DNA, 42 bp, Artificial sequence
  The sequence is synthesized.
gcacacggtgtggtgggcccgcataggccactagtggatctg

SEQ ID NO: 15 - DNA, 20 bp, Artificial sequence
  The sequence is synthesized.
gtgcctctatgatgggtatg

SEQ ID NO: 16 - DNA, 42 bp, Artificial sequence
  The sequence is synthesized.
taccgagctcgaattcgtaataagaacgggaaaccaactatc

SEQ ID NO: 17 - DNA, 39 bp, Artificial sequence
  The sequence is synthesized.
tccactagtggcctatgcaccttgatggatgctctgata

SEQ ID NO: 18 - DNA, 20 bp, Artificial sequence
  The sequence is synthesized.
attcctggagaaccacctaa

SEQ ID NO: 19 - DNA, 42 bp, Artificial sequence
  The sequence is synthesized.
gatagttggtttcccgttcttattacgaattcgagctcggta

SEQ ID NO: 20 - DNA, 40 bp, Artificial sequence
  The sequence is synthesized.
tatcagagcatccatcaaggtgcataggccactagtggat

SEQ ID NO: 21 - DNA, 24 bp, Artificial sequence
  The sequence is synthesized.
tctaactgatctatccaaaactga

SEQ ID NO: 22 - DNA, 19 bp, Artificial sequence
  The sequence is synthesized.
taacgaacgcagaattttc

SEQ ID NO: 23 - DNA, 25 bp, Artificial sequence
  The sequence is synthesized.
atgggcttgttaacgaaagttgcta

SEQ ID NO: 24 - DNA, 25 bp, Artificial sequence
  The sequence is synthesized.
tcaagcatctaaaacacaaccgttg

SEQ ID NO: 25 - DNA, 23 bp, Artificial sequence
  The sequence is synthesized.
atgttgagaactcaagccgccag

SEQ ID NO: 26 - DNA, 23 bp, Artificial sequence
  The sequence is synthesized.
ttattggttttctggtctcaact

SEQ ID NO: 27 - DNA, 17 bp, Artificial sequence
  The sequence is synthesized.
ttcgagctcggtacccg

SEQ ID NO: 28 - DNA, 18 bp, Artificial sequence
  The sequence is synthesized.
agttagaggatccccggg

SEQ ID NO: 29 - DNA, 19 bp, Artificial sequence
  The sequence is synthesized.
cagctgaagcttcgtacgc

SEQ ID NO: 30 - DNA, 22 bp, Artificial sequence
  The sequence is synthesized.
gcataggccactagtggatctg

SEQ ID NO: 31 - DNA, 20 bp, Artificial sequence
  The sequence is synthesized.
gatcatcgtagtgcccaatt

SEQ ID NO: 32 - DNA, 19 bp, Artificial sequence
  The sequence is synthesized.
gtaccgagctcgaattcgt

SEQ ID NO: 33 - DNA, 19 bp, Artificial sequence
  The sequence is synthesized.
ggtttggttgatgcgagtg

SEQ ID NO: 34 - DNA, 21 bp, Artificial sequence
  The sequence is synthesized.
ccattcatcgtgttgttttgg

SEQ ID NO: 35 - DNA, 1613 bp, Artificial sequence
  The sequence is synthesized.
cagctgaagcttcgtacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactagtggcctatgc

(19) United States
(12) Patent Application Publication
(10) Pub. No.: US 2023/0212488 A1
(43) Pub. Date: Jul. 6, 2023

(54) Saccharomyces Uvarum Strain Conductive To Low Production Of Higher Alcohols And Strong Degradation Of Malic Acid And Application Thereof

(71) Applicant: Xinkaitai Technology (Beijing) Co., Ltd., Beijing (CN)

(72) Inventors: Cuiying ZHANG, beijing (CN); Tong Li, beijing (CN); Ping Li, Beijing (CN); Dongguang Xiao, Beijing (CN); Xuewu Guo, Beijing (CN); Liangcai Lin, Beijing (CN); Yefu Chen, Beijing (CN); Aiqun Yu, Beijing (CN)

(21) Appl. No.: 17/061,378

(22) Filed: Oct. 1, 2020

(30) Foreign Application Priority Data
Dec. 5, 2019 (CN) 201911231057.1

(51) Int. Cl. C12G 1/022; C12N 15/81; C12N 9/04

(52) U.S. Cl. CPC C12G 1/0203; C12N 15/81; C12N 9/0006; C12Y 101/0104; C12G 2200/11

(57) ABSTRACT

The present invention provides a Saccharomyces uvarum strain capable of low production of higher alcohols and strong degradation of malic acid. After the wine using Saccharomyces uvarum recombinant strain of the present invention is fermented for 5 days, with other fermentation properties unaffected, the content of isobutanol, isoamyl alcohol and phenethyl alcohol in the wine is 28.18 mg/L, 171.76 mg/L and 13.60 mg/L respectively, which is reduced by 20.28%, 14.77% and 11.26% compared with the starting strain, the total content of main higher alcohols (n-propanol, isobutanol, isoamyl alcohol and phenethyl alcohol) is reduced by 12.97%, and the content of malic acid is reduced to 1.13 g/L after fermentation, which greatly shortens the fermentation period, overcomes the influence of lactic acid bacteria fermentation in the ordinary fermentation process and unpleasant flavor caused by higher content of higher alcohols.

### REFERENCE TO AN ELECTRONIC SEQUENCE LISTING

The contents of the electronic sequence listing (sequence-list-17061378.txt; Size: 18,000 bytes; and Date of Creation: Oct. 21, 2021) is herein incorporated by reference in its entirety.

### CROSS-REFERENCE TO RELATED APPLICATIONS

The application claims the priority of the Chinese patent application filed on Dec. 5, 2019, with the application number of CN201911231057.1 and the invention title of “Saccharomyces Uvarum Strain Capable of Low Production Of Higher Alcohols And Strong Degradation Of Malic Acid And Application Thereof”, the entire contents of which are incorporated herein by reference.

### FIELD OF TECHNOLOGY

The invention belongs to the technical field of bioengineering and genetic engineering, and relates to the breeding and application of industrial microorganisms, in particular relates to a novel Saccharomyces uvarum strain capable of low production of higher alcohols and strong degradation of malic acid and its application in the preparation of wine.

### BACKGROUND

Wine is a kind of low-alcohol fruit wine, brewed with fresh grapes or grape juice as raw materials through full fermentation or partial fermentation. It is a product of harmonious coexistence of mankind with nature. To produce high-quality wine, people cultivate suitable grape varieties under suitable natural conditions over a long period of time to make wine with different flavor characteristics through unique technology. Wine flavor is a comprehensive manifestation of the complementary balance of various flavor substances, and is an important indicator to measure the quality of wine. Higher alcohol is the main by-product of wine fermentation and the main component of wine taste. Cooperation, complementation, foil and restriction between an appropriate amount of higher alcohol and other flavor substances endow wine with special aroma and flavor, giving people a feeling of fruity, full and harmonious.

However, a too high level of higher alcohols not only causes unpleasant off-flavors in the wine, but also causes a toxic effect on the human body due to the slow oxidation rate and long residence time in human body. Therefore, it is necessary to effectively control the content of higher alcohols in the wine-making process.

In the wine-making process, higher alcohols are mainly produced during alcohol fermentation of Saccharomyces ellipsoideus. At present, there have been studies on the breeding of yeast strains through microbial mutation breeding, which can regulate higher alcohols. ZHAI HENG et al. added a certain amount of isoamyl chloroacetate to the yeast culture plate to accurately, conveniently and quickly select yeast strain capable of low production of higher alcohols, which could reduce higher alcohols in wine by 10-15% (Chinese Patent CN103627646B, May 13,2015). XU YAN et al. produced a red wine through a wine-making technology, which had qualified alcoholic volume, meanwhile, the technology could significantly reduce content of higher alcohols and improve the taste of the red wine (Chinese Patent Application CN108060039A, May 22, 2018). Most red wine and some white wine are produced through alcohol fermentation leaded by yeast and malic-lactic fermentation (abbreviated as MLF) leaded by lactic acid bacteria. Malic-lactic fermentation is generally carried out by seeding lactic acid bacteria in the fermentation broth after the alcohol fermentation is completed. It can decarboxylate L-malic acid producing sharp mouthfeel in the wine after alcohol fermentation into L-lactic acid producting soft taste, making the wine mellow and soft.

### SUMMARY

The present invention provides a Saccharomyces uvarum strain capable of low production of higher alcohols and strong degradation of malic acid and application thereof.

The present invention breeds a novel industrial yeast strain capable of low production of high alcohol and strong degradation of malic acid by simultaneously expressing Schizosaccharomyces pombe (S. pombe) mae1 gene and Lactococcus lactis (L. lactis) m1eS gene in Saccharomyces uvarum, The industrial yeast strain can effectively improve the wine flavor quality and greatly shorten the wine fermentation period, bringing significant economic benefits to the wine industry.

The technical solution of the present invention is as follows: The present invention provides a Saccharomyces uvarum strain capable of low production of higher alcohols and strong degradation of malic acid, which simultaneously heterologously expresses Schizosaccharomyces pombe mae1 gene and Lactococcus lactis m1eS gene.

The mae1 gene has a Gene ID of 2543334, with a nucleotide sequence as shown in SEQ NO: 1 in the Sequencing Listing; the m1eS gene has a Gene ID of 1114530, with a nucleotide sequence as shown in SEQ NO: 2 in the Sequencing Listing.

The Gal80 gene has a Gene ID of 854954, with a nucleotide sequence as shown in SEQ NO: 3 in the Sequencing Listing. The promoter PGK1 has a Gene ID of 850370, with a nucleotide sequence as shown in SEQ NO: 4 in the Sequencing Listing.

The starting yeast strain is Saccharomyces uvarum CICC1465.

The present invention provides a method for constructing a Saccharomyces uvarum strain capable of low production of higher alcohol and strong degradation of malic acid, including the following steps:

(1) Construction of recombinant fragments heterologously expressing mae1 gene and m1eS gene.

\textcircled{1} Using a plasmid pPGK1 as a template, the PGK1 gene fragment is amplified by PCR, the strong promoter PGK1 as a PCR product is recovered, and a plasmid Yep352 and the PGK1 fragment are subjected to double digestion with BamHI and SalI simultaneously and then ligated together to construct a plasmid Yep-P.

\textcircled{2} Using the genome of the starting yeast strain as a template, the mae1 and m1eS genes are separately amplified by PCR, the plasmid Yep-P is digested with a restriction enzyme XhoI, and the gene fragments of mae1 and m1eS are separately ligated with the plasmid Yep-P to construct plasmid Yep-Pm1 and plasmid Yep-PS.

\textcircled{3} Using the plasmid Yep-PS as a template, a PGK1p-m1eS-PGK1t fragment is amplified by PCR. The plasmid Yep-Pm1 is digested with the restriction enzyme SmaI, and the fragment PGK1p-m1eS-PGK1t is ligated with the plasmid Yep-Pm1 to construct a plasmid Yep-Pm1S.

\textcircled{4} Using a plasmid pUG6 as a template, a KanMX gene is amplified by PCR, the plasmid Yep-Pm1 S is digested with the restriction enzyme ApaI, and then ligated with KanMX gene fragment to construct a plasmid Yep-KPm1S.

(2) Construction of a recombinant strain expressing mae1 and m1eS genes

\textcircled{1} Using the plasmid Yep-Pm1SK as a template to amplify PGK1-mae1-PGK1-m1eS-KanMX gene containing Gal80 upstream homologous arm and downstream homologous arm genes by PCR.

\textcircled{2} Introducing the PCR product of step \textcircled{1} into the starting strain CICC1465 to obtain a recombinant strain WY-m1S which simultaneously overexpresses mae1 and m1eS genes.

The present invention also provides application of the above-mentioned strain in preparing wine.

Preferably, the wine has low content of higher alcohols and malic acid.

The beneficial effects of the present invention are as follows:

1. The Saccharomyces uvarum strain capable of low production of higher alcohols and strong degradation of malic acid provided by the present invention simultaneously expresses S. pombe mae1 gene and L. lactis m1eSgene on the premise of maintaining good fermentation performance, which achieves the purpose of simultaneously regulating higher alcohols and malic acid and lays a theoretical foundation for brewing wine with excellent flavor and short fermentation period.

2. After the wine using Saccharomyces uvarum recombinant strain of the present invention is fermented for 5 days, with other fermentation properties are not affected, the content of isobutanol, isoamyl alcohol and phenethyl alcohol in the wine is 28.18 mg/L, 171.76 mg/L and 13.60 mg/L respectively, which is reduced by 20.28%, 14.77% and 11.26% as compared with the starting strain, the total content of main higher alcohols (n-propanol, isobutanol, isoamyl alcohol, and phenethyl alcohol) is reduced by 12.97%, and the content of malic acid is reduced to 1.13 g/L after fermentation, which eliminates the influence of lactic acid bacteria fermentation and greatly shortens the fermentation period.

3. The Saccharomyces uvarum recombinant strain of the present invention overcomes the problems of inharmonious flavor due to the high content of higher alcohols of ordinary yeast and prolonged fermentation period due to lactic acid bacteria fermentation, improves the flavor quality of wine and shortens fermentation period, so that it has promising market prospects.

### BRIEF DESCRIPTION OF THE DRAWINGS

FIG. 1 shows the construction process of the Yep-KPm1S plasmid;

FIG. 2 shows a verification diagram of the plasmids Yep-Pm1, Yep-PS, Yep-Pm1S and Yep-KPm1S and recombinant strain WY-m1S;

wherein M is a marker; lane 1 is the result of PCR amplification using Yep352 as a template and YP-F/YP-R as a primer; lane 2 is the PGK1 gene fragment amplified by PCR using Yep-P as a template and YP-F/YP-R as a primer; lane 3 is result of PCR amplification using Yep-P as a template and Ymae1-F/Ymae1-R as a primer; lane 4 is the mae1 gene fragment amplified by PCR using Yep-Pm1 as a template and Ymae1-F/Ymae1-R as a primer; lane 5 is the result of PCR amplification using Yep-P as a template and Ym1eS-F/Ym1eS-R as a primer; lane 6 is the m1eS gene fragment amplified by PCR using Yep-PS as a template and Ym1eS-F/Ym1eS-R as a primer; lane 7 is the result of PCR amplification using Yep-Pm1 as a template and SmaI-F/SmaI-R as a primer; lane 8 is the PGK1p-m1eS-PGK1t fragment amplified by PCR using Yep-Pm1S as a template and SmaI-F/SmaI-R as a primer; lane 9 is the result of PCR amplification using Yep-Pm1S as a template and YK-F/YK-R as a primer; lane 10 is the KanMX gene fragment amplified by PCR using Yep-KPmS as a template and YK-F/YK-R as a primer.

FIG. 3a shows a verification diagram of the plasmids Yep-Pm1, Yep-PS, Yep-Pm1S, Yep-KPm1 S and the recombinant strain WY-m1 S, wherein M is a marker; 1 and 2 are verification fragments amplified by PCR using the DNA of the starting strain CICC1465 and the recombinant strain WYm1S respectively as a template and YA-F/YA-R as a primer.

FIG. 3b shows the verification diagram of plasmids Yep-Pm1, Yep-PS, Yep-Pm1S, Yep-KPm1 S and recombinant strain WY-m1 S, wherein M is a marker; 1 and 2 are verification fragments amplified by PCR using the DNA of the starting strain CICC1465 and the recombinant strain WYm1S respectively as a template and YB-F/YB-R as a primer.

### DESCRIPTION OF THE EMBODIMENTS

Saccharomyces uvarum is a non-Saccharomyces yeast with potential wine-making properties, and produces more aromatic substances than Saccharomyces cerevisiae. Constructing Saccharomyces uvarum industrial strains that simultaneously regulate malic acid and higher alcohols by using molecular breeding techniques is of great significance to shorten the wine fermentation period and improve the flavor quality of wine.

Malic-lactic fermentation is generally carried out by seeding lactic acid bacteria in the fermentation broth after the alcohol fermentation is completed. It can decarboxylate L-malic acid producing sharp mouthfeel in the wine after alcohol fermentation into L-lactic acid producting soft taste, making the wine mellow and soft. However, after the alcohol fermentation is completed, the high alcoholic strength, low pH value and content of residual sugar of the wine body will inhibit the normal metabolism of lactic acid bacteria, which will hinder the fermentation. Also, the existence of bacteriophage in the wine body will also delay or inhibit malic-lactic fermentation, and fermentation of spoilage bacteria produces peculiar smell, leading to the occurrence of wine diseases and reducing the flavor quality of wine. Therefore, in the wine-making process, construction of yeast strains that simultaneously regulate higher alcohols and strongly degrade malic acid by industrial microbial breeding is an essential approach to solve the problems of high content of higher alcohols and prolonged wine fermentation period due to lactic acid bacteria fermentation.

The present invention will be described below through specific embodiments. Unless otherwise specified, the technical means used in the present invention are all methods known to those skilled in the art. In addition, the embodiments should be understood as illustrative rather than limiting the scope of the present invention, and the essence and scope of the present invention are defined only by the claims. For those skilled in the art, without departing from the essence and scope of the present invention, various changes or modifications to the material components and amounts in these embodiments also belong to the protection scope of the present invention.

The Saccharomyces uvarum strain capable of low production of higher alcohols and strong degradation of malic acid was obtained by simultaneously integrating S. pombe mae1 gene and L. lactis m1eS gene into the Gal80 gene locus of the starting Saccharomyces uvarum strain under the regulation of promoter PGK1 and with using KanMX gene as the selection marker.

Starting strain used in this embodiment was CICC1465. The Escherichia co/i DH5a was purchased from Takara, and S. pombe CICC1757 and L. lactis NZ9000 were purchased from the China Center of Industrial Culture Collection.

The YPD medium was a universal complete medium, and the solid medium contained 2% imported agar powder.

The mae1 gene had a Gene ID of 2543334 with a nucleotide sequence as shown in SEQ NO: 1 in the Sequencing Listing. The m1eS gene had a Gene ID of 1114530 with a nucleotide sequence as shown in SEQ NO: 2 in the Sequencing Listing. The Gal80 gene had a Gene ID of 854954 with a nucleotide sequence as shown in SEQ NO: 3 in the Sequencing Listing. The promoter PGK1 had a Gene ID of 850370 with a nucleotide sequence as shown in SEQ NO: 4 in the Sequencing Listing. The nucleotide sequence of KanMX gene was as shown in SEQ ID NO: 5 in the Sequencing Listing.

Based on the yeast genome data in Genebank and the integrated plasmid sequence, the following primers were designed.

TABLE 1
Primers used in this example

| | | Restriction | SEQ |
|---|---|---|---|
| | | Enzyme | ID |
| Primer | Sequence (5′→3′) | cutting site | NO |
| PGK-F | CGCGGATCCTCTAACTGATCTATCCAAAACT | BamHI | 5 |
| | G | | |
| PGK-R | ACGCGTCGACTAACGAACGCAGAATTTTCG | SalI | 6 |
| | AG | | |
| mael-F | GAATTCCAGATCTCCTCGAGTTCATTTTCTC | | 7 |
| | TCTTGGC CAC | | |
| mael-R | TCTATCGCAGATCCCTCGAGCTTTTGTCATG | | 8 |
| | AAATCCC TCTTA | | |
| mleS-F | GAATTCCAGATCTCCTCGAGATGCGTGCAC | | 9 |
| | ATGAAAT TT | | |
| mleS-R | TCTATCGCAGATCCCTCGAGTTAGTACTCTG | | 10 |
| | GATACCA TTTAAGA | | |
| PGK_{(SmaI)}-F | CGGCCCGGGTCTAACTGATCTATCCAAAA | SmaI | 11 |
| PGK_{(SmaI)}-R | CGGCCCGGGTAACGAACGCAGAATTTTCG | SmaI | 12 |
| K-F | CCGCTAACAATACCTGGGCCCCAGCTGAAG | | 13 |
| | CTTCGT | | |
| | ACGC | | |
| K-R | GCACACGGTGTGGTGGGCCCGCATAGGCCA | | 14 |
| | CTAGTG | | |
| | GATCTG | | |
| A-F | GTGCCTCTATGATGGGTATG | | 15 |
| A-R | TACCGAGCTCGAATTCGTAATAAGAACGGG | | 16 |
| | AAACCA ACTATC | | |
| B-F | TCCACTAGTGGCCTATGCACCTTGATGGATG | | 17 |
| | CTCTGATA | | |
| B-R | ATTCCTGGAGAACCACCTAA | | 18 |
| mS-F | GATAGTTGGTTTCXXGTTCTTATTACGAATT | | 19 |
| | CGAGCTCGGTA | | |
| mS-R | TATCAGAGCATCCATCAAGGTGCATAGGCC | | 20 |
| | ACTAGTG GAT | | |
| YP-F | TCTAACTGATCTATCCAAAACTGA | | 21 |
| YP-R | TAACGAACGCAGAATTTTC | | 22 |
| Ymael-F | ATGGGCTTGTTAACGAAAGTTGCTA | | 23 |
| Ymael-R | TCAAGCATCTAAAACACAACCGTTG | | 24 |
| YmlcS-F | ATGTTGAGAACTCAAGCCGCCAG | | 25 |
| YmlcS-R | TTATTGGTTTTCTGGTCTCAACT | | 26 |
| Smal-F | TTCGAGCTCGGTACCCG | | 27 |
| Smal-R | AGTTAGAGGATCCCCGGG | | 28 |
| YK-F | CAGCTGAAGCTTCGTACGC | | 29 |
| YK-R | GCATAGGCCACTAGTGGATCTG | | 30 |
| YA-F | GATCATCGTAGTGCCCAATT | | 31 |
| YA-R | GTACCGAGCTCGAATTCGT | | 32 |
| YB-F | GGTTTGGTTGATGCGAGTG | | 33 |
| YB-R | CCATTCATCGTGTTGTTTTGG | | 34 |

Note:
what is underlined is the restriction site.

TABLE 2
The PCR amplification system used in this example

| Reaction system | Loading amount |
|---|---|
| ddH₂O | Made up to 50 μL |
| 10 × PCR Buffer | 5.0 μL |
| dNTP (0.2 mmol/L) | 4 μL |
| Upstream and downstream | each 1.5 μL |
| primers (10 m moI/L) | |
| Template: Yeast’s total DNA | 1.0 μL |
| LA-Taq DNA polymerase | 0.25 μL |

### Embodiment 1

Construction of Saccharomyces uvarum Overexpressing Mae1 and m1eS.

(1) Construction of Recombinant Plasmid Yep-KPm1S

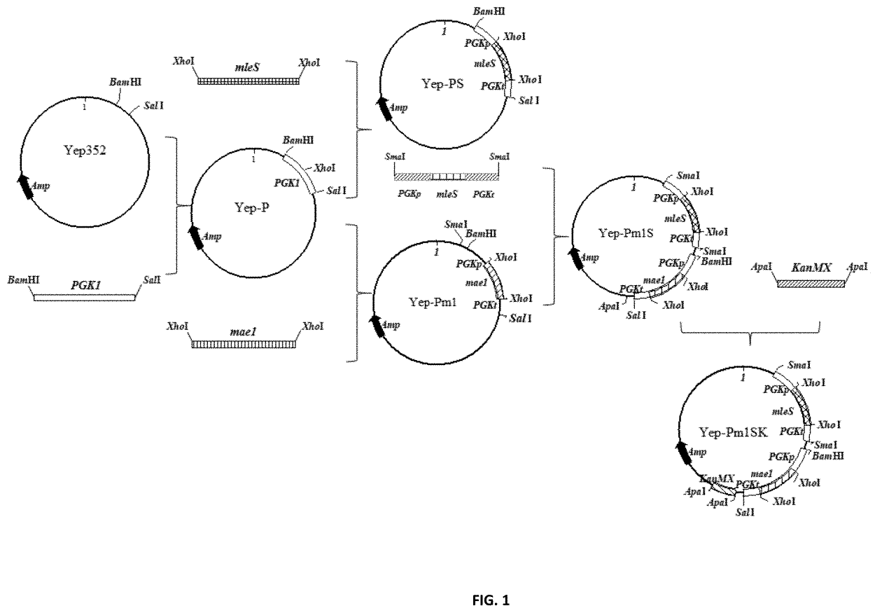

The construction process of the recombinant plasmid Yep-Pm1 is shown in FIG. 1;

Plasmid pPGK1 was used as a template, PGK-F (SEQ ID NO: 5) and PGK-R (SEQ ID NO: 6) were used as primers, the PGK1 gene fragment (SEQ ID NO: 4) was amplified by PCR, with the PCR reaction conditions being as follows: 95° C. for 5 min; 94° C. for 40 s, 56° C. for 1 min, 72° C. for 108 s, 30 cycles; 72° C. for 10 min. Plasmid Yep532 and PGK1 gene fragments were digested with restriction enzymes BamHI and SalI, and then ligated to construct a plasmid Yep-P. The genome of S. pombe CICC1757 strain was used as a template, mae1-F (SEQ ID NO:7) and mae1-R (SEQ ID NO: 8) were used as primers, PCR amplification was conducted to obtain mae1 fragment, (SEQ ID NO: 1), with the PCR reaction conditions being as follows: 95° C. for 5 min; 94° C. for 40 s, 56° C. for 1 min, 72° C. for 108 s, 30 cycles; 72° C. for 10 min. The fragment is ligated with Yep-P plasmid digested with XhoI through homologous recombination to construct plasmid Yep-Pm1. The genome of L. lactis NZ9000 strain was used as a template, and m1eS-F (SEQ ID NO: 9) and m1eS-R (SEQ ID NO: 10) were used as primers, PCR amplification was conducted to obtain fragment m1eS (SEQ ID NO: 2), with the PCR reaction conditions being as follows: 95° C. for 5 min; 94° C. for 40 s, 56° C. for 1 min, 72° C. for 108 s, 30 cycles; 72° C. 10 min. The resultant fragment and the Yep-P plasmid digested with XhoI were ligated by homologous recombination to construct a plasmid Yep-PS. The plasmid Yep-PS was used as a template, and PGK_{(smaI)}-F (SEQ ID NO: 11) and PGK_{(smaI)}-R (SEQ ID NO: 12) were used as primers, PCR amplification was conducted to obtain a fragment PGK1p-m1eS-PGK1t, with PCR reaction conditions being as follows: 95° C. for 5 min; 94° C. for 40 s, 56° C. for 1 min, 72° C. for 108 s, 30 cycles; 72° C. for 10 min. The fragment was ligated with Yep-Pm1 plasmid digested with SmaI through homologous recombination to construct a plasmid Yep-Pm1S. The plasmid pUG6 (nucleotide sequence as shown in SEQ ID NO:35) was used as a template, K-F (SEQ ID NO: 13) and K-R (SEQ ID NO: 14) were used as primers, the selection marker KanMX gene fragment was amplified by PCR, with the PCR reaction conditions being as follows: 95° C. for 5 min; 94° C. for 40 s, 57° C. for 1 min, 72° C. for 100 s, 30 cycles; 72° C. for 10 min. The plasmid Yep-Pm1S was digested with the restriction enzyme ApaI and then ligated with the KanMX gene fragment through homologous recombination to construct a plasmid Yep-KPm1S.

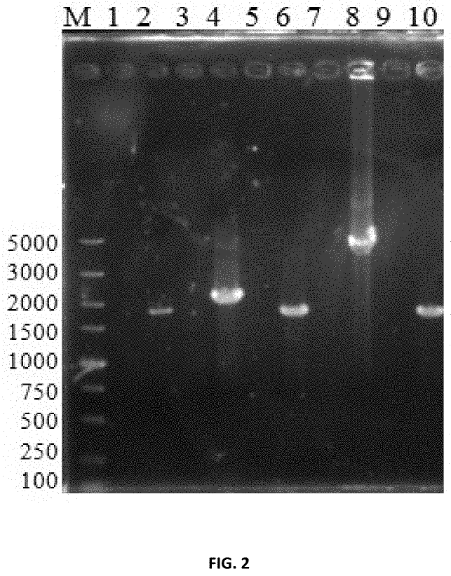

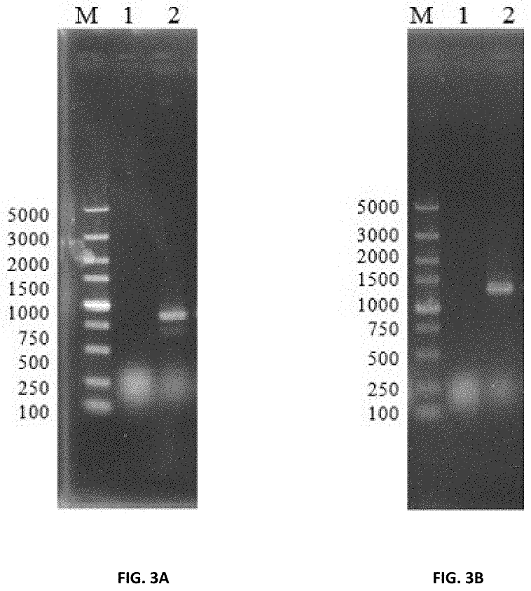

The PCR verification results are shown in FIG. 2, wherein M is a marker; lane 1 is the result of PCR amplification with Yep352 as a template and YP-F (SEQ ID NO: 21) and YP-R (SEQ ID NO: 22) as primers. Lane 2 is the PCR-amplified PGK1 gene fragment with Yep-P as a template, and YP-F (SEQ ID NO: 21) and YP-R (SEQ ID NO: 22) as primers, and the plasmid Yep-P can be amplified by PCR to obtain PGK1 gene fragment, but Yep352 cannot, indicating that gene PGK1 has been successfully ligated with plasmid Yep352, and the plasmid Yep-P is successfully constructed; lane 3 is the result of PCR amplification with Yep-P as a template, and Ymae1-F (SEQ ID NO: 23) and Ymae1-R (SEQ ID NO: 24) as primers, lane 4 is the PCR-amplified mae1 genes fragment with Yep-Pm1 as a template, and Ymae1-F (SEQ ID NO: 23) and Ymae1-R (SEQ ID NO: 24) as primers, and plasmid Yep-Pm1 can be amplified by PCR to obtain the mae1 gene fragment, but Yep-P cannot, indicating that the gene mae1 has been successfully ligated with plasmid Yep-P, and the plasmid Yep-Pm1 is successfully constructed; lane 5 is the result of PCR amplification with Yep-P as a template, and Ym1eS-F (SEQ ID NO: 25) and Ym1eS-R (SEQ ID NO: 26) as primers, lane 6 is the PCR-amplified m1eS gene fragment with Yep-PS as a template, and Ym1eS-F (SEQ ID NO: 25) and Ym1eS-R (SEQ ID NO: 26) as primers. The plasmid Yep-PS can be amplified by PCR to obtain the m1eS gene fragment, but Yep-P cannot, indicating that the gene m1eS has been successfully ligated with plasmid Yep-P, and Yep-PS is successfully constructed; lane 7 is the result of PCR amplification with Yep-Pm1 as a template, and SmaI-F (SEQ ID NO: 27) and SmaI-R (SEQ ID NO: 28) as primers, lane 8 is the PCR-amplified fragment with Yep-Pm1S as a template, and SmaI-F (SEQ ID NO:27) and SmaI-R (SEQ ID NO:28) as primers, and the plasmid Yep-Pm1S can be amplified by PCR to obtain the PGKp-m1eS-PGKt gene fragment, but Yep-Pm1 cannot, indicating that the gene fragment PGKp-m1eS-PGKt has been successfully ligated with the plasmid Yep-Pm1, and the plasmid Yep-Pm1S is successfully constructed. Lane 9 is the result of PCR amplification with Yep-Pm1 S as a template, and YK-F (SEQ ID NO: 29) and YK-R (SEQ ID NO: 30) as primers, lane 10 is the PCR-amplified KanMX gene fragment with Yep-Pm1SK as a template, and YK-F (SEQ ID NO: 29) and YK-R (SEQ ID NO: 30) as primers, and plasmid Yep-Pm1SK can be amplified by PCR to obtain the KanMX gene fragment, but Yep-Pm1 S cannot, indicating that the gene fragment KanMX has been successfully ligated with plasmid Yep-Pm1S, and the plasmid Yep-Pm1SK is successfully constructed.

(2) Construction of Recombinant Strain WYm1S

The plasmid Yep-KPm1S was used as a template, mS-F (SEQ ID NO: 19) and mS-R (SEQ ID NO: 20) were used as primers, the gene fragment A-PGKp-mae1-PGKt-PGKp-m1eS-PGKt-KanMX-B containing gene Gal80 upstream and downstream homologous arms was amplified by PCR, with the PCR reaction conditions being as follows: 95° C. for 5 min; 94° C. for 40 s, 56° C. for 1 min, 72° C. for 108 s, 30 cycles; 72° C. for 10 min.

The PCR product was transferred into the starting strain CICC1465 by the lithium acetate conversion method, the recombinant strain WYm1S was selected by G418 resistance, the genomes of the recombinant strain and the starting strain CICC1465 were extracted, primers YA-F (SEQ ID NO: 31) and YB-R (SEQ ID NO: 34) were designed exterior the upstream and downstream of the Gal80 gene respectively, and primers YA-R (SEQ ID NO: 32) and YB-F (SEQ ID NO: 33) were designed in the gene fragment PGKp-mae1-PGKt-PGKp-m1eS-PGKt-KanMX. PCR was performed using each genome as a template and YA-F (SEQ ID NO: 31)/YA-R (SEQ ID NO: 32) as primers. The recombinant strain WYm1S genome could be amplified to obtain a fragment of about 860 bp in size, which is consistent with the expected size of the target product, whereas the starting strain could not be amplified to obtain the corresponding fragment. The PCR verification results are shown in FIG. 3(a), wherein M is a marker, lane 1 is the result of PCR amplification using the starting strain CICC1465 as a template, and YA-F (SEQ ID NO: 31) and YA-R (SEQ ID NO: 32) as primers, lane 2 is the PCR-amplified gene fragment using WYm1S as a template, and YA-F (SEQ ID NO: 31) and YA-R (SEQ ID NO:32) as primers. For PCR using YB-F (SEQ ID NO: 33)/YB-R (SEQ ID NO: 34), the recombinant strain WYm1S genome could be amplified to obtain a fragment of about 1400 bp in size, which is consistent with the expected size of the target product, whereas the starting strain could not be amplified to obtain the corresponding fragment. The PCR verification results are shown in FIG. 3(b), wherein M is a marker, lane 1 is the result of PCR amplification using the starting strain CICC1465 as a template, and YB-F (SEQ ID NO: 33)/YB-R (SEQ ID NO: 34) as primers, and lane 2 is the PCR-amplified gene fragment using WYm1S as a template, and YB-F (SEQ ID NO: 33)/YB-R (SEQ ID NO:34) as primers, indicating that the gene fragment PGKp-mae1-PGKt-PGKp-m1eS-PGKt-KanMX has been successfully integrated into the position of gene Gal80, and the strain WYm1S was successfully constructed.

### Embodiment 2

Fermentation Experiment of Saccharomyces uvarum Strain Capable of Low Production of Higher Alcohols

(1) Wine Fermentation Experiment of Recombinant Strain and Starting Strain

\textcircled{3} Fermentation process route: Grape raw material: select, clean, dry, destem; crush; adjust sugar, adjust acid; add sulfurous acid, sterilize; inoculate; preliminarily ferment; separate dreg; measure parameters.

\textcircled{4} Process conditions: Sugar degree: 20.45 Brix; Acidity: pH 3.5; SO₂ content: 80 mg/L, standing at 4° C. for 12 h; Liquid volume in flask: 190 mL grape juice in a 250 mL triangular flask; inoculation amount: 1×10⁸ CFU/mL; fermentation temperature and time: 25° C., 5 d; steaming conditions: 100 mL fermentation broth steamed with 100 mL water to obtain 100 mL wine sample.

According to the above fermentation process, Saccharomyces uvarum starting strain CICC1465 and the strain WY-m1S of the Embodiment 1 were used for wine fermentation experiment. Shaking and weighing were performed every 12 hours during the fermentation, and the weight loss was recorded. After the fermentation was completed, the cultivation was stopped and weighing was conducted. The temperature and alcoholic strength of the distillate were measured by a thermometer and an oenometer respectively, and the alcoholic strength at this temperature was converted to the corresponding alcoholic strength at 20° C. The reducing sugar content in the wine was determined using the fehlings reagent method, and the results are shown in Table 3. Table 3 shows that in the wine fermentation experiment, the basic fermentation performance of the Saccharomyces uvarum recombinant strain WY-m1S obtained by the present invention is not much changed compared with the starting strain CICC1465.

TABLE 3
Determination of fermentation performance of
starting strain and recombinant strain

| | Weight | Residual | Alcoholic |
|---|---|---|---|
| Strain | loss (g) | sugar (g/L) | strength (% vol) |
| CICC1465 | 14.95 | 1.94 | 11.20 |
| WYmlS | 15.03 | 2.08 | 11.18 |

Note:
The data shown are the average of three parallel test results.

(2) Contents Determination of Malic Acid and Higher Alcohols

The contents of malic acid and higher alcohols in the wine after fermentation were determined by high-performance liquid chromatography (HPLC) and gas chromatography (GC). HPLC analysis: the wine fermentation broth was filtered by a 0.22 μm fiber filter membrane and then analyzed by high-performance liquid chromatography, with the chromatographic conditions being as follows: the column is Bio-RadHIPX-87H, 300×7.8 mm; the detector was a differential refractive index detector (RID); the mobile phase was 5 mmol/L sulfuric acid, the flow rate was 0.6 mL/min; the detector temperature was 45° C., the column temperature was 65° C., and the injection volume was 20 μl. GC analysis: after the fermentation broth was distilled, the wine sample was analyzed by high-performance gas chromatography, with the chromatographic conditions being as follows: the gas chromatograph is Agilent 7890C, and was equipped with the Agilent G4512A automatic sampler, the column was Agilent 1909N-213, 30 m×0.32 mm×0.5 m capillary column, the detector is FID. The inlet temperature was set to 200° C. and the detector temperature was 200° C. Injection volume condition: 1 μL injection volume, and split ratio of 5:1. The carrier gas was high-purity nitrogen, and the flow rate was set to be 2.0 mL/min. Heating program: the initial column temperature was set to be 50° C. and held for 8 min, and then increased to 120° C. at a heating rate of 5° C./min and kept for 5 min. The results are shown in Table 4.

Table 4 shows that the content of isobutanol, isoamyl alcohol and phenylethanol in the wine after fermentation with the recombinant strain WY-m1S is 28.184 mg/L, 171.756 mg/L and 13.604 mg/L, respectively, which is reduced by 20.28%, 14.77% and 11.26% as compared with the starting strain, the total content of higher alcohols (isobutanol, isoamyl alcohol, phenethyl alcohol) is 213.54 mg/L, which is reduced by 15.33% as compared with the starting strain.

Moreover, the content of malic acid obtained with the recombinant strain WYm1S of the present invention reaches 1.130 mg/L, which is almost consistent with the content of malic acid in wine fermented with lactic acid bacteria. This shows that the strain obtained by the present invention can greatly reduce the content of higher alcohols in wine, and can effectively degrade malic acid during alcohol fermentation, thereby eliminating the effect of lactic acid bacteria fermentation, and greatly shortening the wine fermentation period. Meanwhile, it provides a theoretical basis for enriching the taste of wine and improving the flavor quality of wine.

TABLE 4
Content of malic acid and higher alcohols for the
starting strain and the recombinant strain (mg/L)

| | Malic | n- | | Isoamyl | Phenethyl |
|---|---|---|---|---|---|
| | acid | propanol | Isobutanol | alcohol | alcohol |
| Strain | (g/L) | (mg/L) | (mg/L) | (mg/L) | (mg/L) |
| CICC1465 | 3.681 | 51.830 | 35.354 | 201.530 | 15.330 |
| WYmlS | 1.130 | 51.068 | 28.184 | 171.756 | 13.604 |

Note:
The data shown is the average of three parallel test results.

We claim:

1. A Saccharomyces uvarum strain capable of low production of higher alcohols and strong degradation of malic acid, wherein, heterologously expressed Schizosaccharomyces pombe mae1 gene and Lactococcus lactis m1eS gene are introduced into Saccharomyces uvarum CICC1465.

2. The Saccharomyces uvarum strain capable of low production of higher alcohols and strong degradation of malic acid according to claim 1, wherein, the mae1 gene has a nucleotide sequence as shown in SEQ ID NO: 1; and the m1eS gene has a nucleotide sequence as shown in SEQ ID NO: 2.

3. The Saccharomyces uvarum strain capable of low production of higher alcohols and strong degradation of malic acid according to claim 1, wherein, PGK1 gene is used as a promoter, and the promoter PGK1 gene has a nucleotide sequence as shown in SEQ ID NO: 4.

4. The Saccharomyces uvarum strain capable of low production of higher alcohols and strong degradation of malic acid according to claim 3, wherein, a KanMX gene is a selection marker, the mae1 gene and the m1eS gene are simultaneously integrated into Gal80 gene locus under the regulation of the promoter PGK1.

5. The Saccharomyces uvarum strain capable of low production of higher alcohols and strong degradation of malic acid according to claim 4, wherein, the KanMX gene has a nucleotide sequence as shown in SEQ ID NO: 5.

6. A method for constructing a Saccharomyces uvarum strain capable of low production of higher alcohols and strong degradation of malic acid, characterized by comprising the following steps:
(1) construction of recombinant fragments
\textcircled{1} ligating the promoter gene PGK1 to BamHI and SalI cleavage sites of a plasmid Yep352 to construct a plasmid Yep-P;
\textcircled{2} integrating gene fragments of mae1 gene and m1eS gene separately into the plasmid Yep-P at an XhoI site of the gene PGK1 by homologous recombination to construct plasmids Yep-Pm1 and Yep-PS;
\textcircled{3} ligating a PGKp-m1eS-PGKt gene fragment of the plasmid Yep-PS with the plasmid Yep-Pm1 through SmaI digestion to construct a plasmid Yep-Pm1S;
\textcircled{4} integrating a gene fragment KanMX used as selection marker into an ApaI site of the plasmid Yep-Pm1 S by homologous recombination to construct a plasmid Yep-Pm1 SK;
(2) construction of a recombinant strain heterologously expressing mae1 gene and m1eS gene
\textcircled{1} using the plasmid Yep-Pm1SK as a template to amplify PGK1-mae1-PGK1-m1eS-KanMX gene containing Gal80 upstream homologous arm and downstream homologous arm genes by PCR;
\textcircled{2} introducing a PCR product of the PGK1-mae1-PGK1-m1eS-KanMX gene into the Saccharomyces uvarum CICC1465 to obtain a recombinant strain WYm1S capable of simultaneously expressing mae1 and m1eS genes.

7. An application of the Saccharomyces uvarum strain capable of low production of higher alcohols and strong degradation of malic acid according to claim 1 in wine fermentation.
